(19) United States
(12) Patent Application Publication
(10) Pub. No.: US 2024/0092787 A1
(43) Pub. Date: Mar. 21, 2024

(54) PHARMACEUTICAL PROCESS AND INTERMEDIATES

(71) Applicant: ASTRAZENECA AB, Södertälje (SE)

(72) Inventors: MARK Andrew GRAHAM, CAMBRIDGE (GB); JONATHAN ROBERT CARNEY, CAMBRIDGE (GB); THOMAS GEORGE BISHOP, CAMBRIDGE (GB); KATIE COOPER, CAMBRIDGE (GB)

(21) Appl. No.: 18/452,008

(22) Filed: Aug. 18, 2023

Related U.S. Application Data
(60) Provisional application No. 63/400,445, filed on Aug. 24, 2022.

(51) Int. Cl. C07D 487/04; B01J 31/22; B01J 31/24; C07D 207/34

(52) U.S. Cl. CPC C07D 487/04; B01J 31/2226; B01J 31/2409; C07D 207/34; B01J 2231/4277; B01J 2531/004; B01J 2531/824; C07B 2200/13

(57) ABSTRACT

There is provided a new and improved synthetic route for the synthesis of the compound 1-{2-[(1R)-1-aminoethyl]-4-chlorobenzyl}-2-thioxo-1,2,3,5-tetrahydro-4H-pyrrolo[3,2-d]pyrimidin-4-one (Formula (I)) that is readily scalable for commercial production.

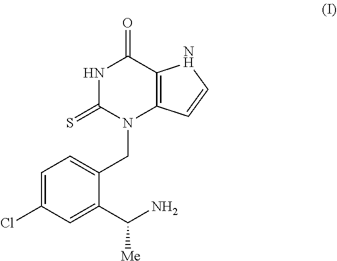 [SMILES: C[C@@H](N)c1cc(Cl)ccc1Cn1c(=S)[nH]c(=O)c2[nH]ccc21]

Also provided are important intermediate compounds that are formed in the new and improved synthetic route for the synthesis of the compound of formula (I).

### RELATED APPLICATIONS

The application claims the benefit of priority to U.S. patent application Ser. No. 63/400,445, filed on 24^{th} Aug., 2022, which is incorporated herein by reference in its entirety.

International Patent Application WO2016/087338 discloses thioxopyrrolopyrimidone derivatives useful as inhibitors of myeloperoxidase (MPO). In particular, discloses 1-{2-[(1R)-1-aminoethyl]-4-chlorobenzyl}-2-thioxo-1,2,3,5-tetrahydro-4H-pyrrolo[3,2-d]pyrimidin-4-one (hereafter referred to as the compound of formula (I)). The structure of the compound of formula (I) is shown below.

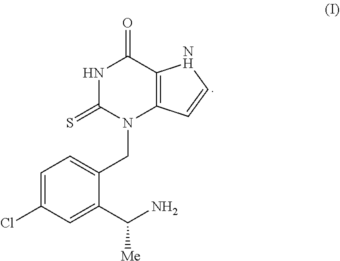 [SMILES: C[C@@H](N)c1cc(Cl)ccc1Cn1c(=S)[nH]c(=O)c2[nH]ccc21]

The compound of formula (I) is being developed as an active pharmaceutical compound for the treatment of respiratory diseases. Appropriate methods for safe, cost-effective, efficient and environmentally sensitive manufacture of the compound of formula (I) may therefore be desirable.

Previous enantioselective syntheses of the compound of Formula (I) are described in Example 3 of WO2016/087338.

One previous synthesis involves 10 steps (a-j) at 22% overall yield and provides 633 g of the compound. The yield given for step (i) of this synthesis is on a sub-gram scale. Dioxane protection/deprotection of an acetophenone was required.

Another previous synthesis (alternative Example 3) involves at least 8 steps at 47% yield and provides 16 g of the compound (note the reductive amination and cyclisation steps are implicitly performed between (e) and (f)). BOC protection/deprotection of an amine was required.

Both previous syntheses rely on dichloromethane, a less environmentally friendly and carcinogenic solvent. Both previous syntheses required the use of toxic carbon monoxide gas (one also uses flammable hydrogen gas) and above atmospheric pressures to introduce a formyl group. Such reaction conditions do not scale well. Both previous syntheses also perform the enantioselective amine formation first which requires an expensive chiral sulfinamide in stoichiometric amounts. The loss of material in the subsequent steps of these syntheses is therefore less cost effective than if the enantioselective amine formation was performed later in the synthesis.

### SUMMARY OF THE INVENTION

The present invention relates to a new and improved synthetic route for the synthesis of the compound of formula (I) that is readily scalable for commercial production and is described below in Scheme 1. The square brackets indicate compounds that may be telescoped through to the next step in their crude form (i.e. without isolation and/or purification). Compound (Ia1) is pictured as the hydrochloride salt, but the corresponding freebase or a different salt thereof may be used.

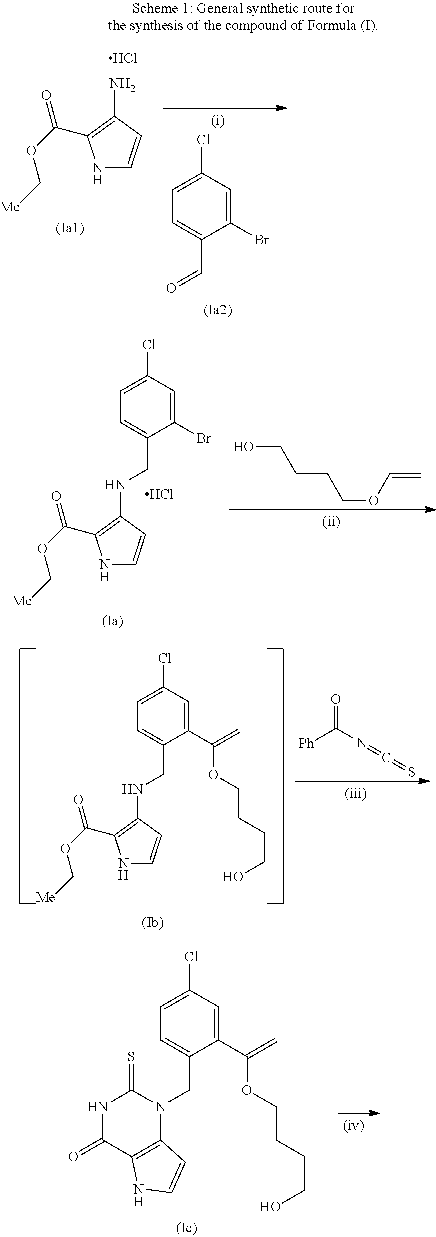

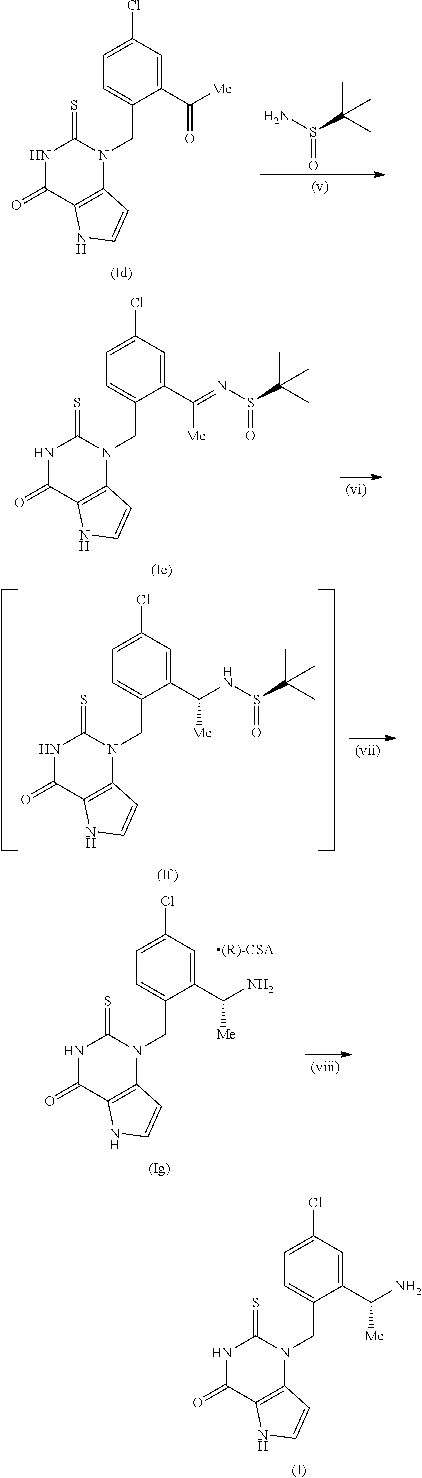

In a first aspect there is provided a process for preparing a compound of Formula (I) comprising at least one of the steps (i) to (viii).

The new and improved process involves only 8 steps at 46% overall yield and is commercially scalable to provide at least 82 kg of the compound (over 130 times more by weight than the largest previous synthesis). It may even be considered only 6 steps if the two telescope procedures described herein are performed.

Chlorinated solvents are not used. The need for a protection step in the main reaction sequence is dispensed with, because the only protecting group is introduced as part of the acetophenone formation and removed after the cyclisation step. Unlike both previous syntheses, the enantioselective amine formation is the last structural transformation and so the resulting valuable product is maximally conserved for cost effectiveness. Furthermore, the formylation step is avoided because a commercially available starting material already comprises the formyl group necessary for the reductive amination.

In one embodiment, the process for preparing a compound of Formula (I) comprises at least two, three, four, five, six, seven or all of steps (i) to (viii).

In one embodiment, the process for preparing a compound of Formula (I) comprises at least two of steps (i) to (viii), optionally (i) and (ii); (i) and (iii); (i) and (iv); (i) and (v); (i) and (vi); (i) and (vii); (i) and (viii); (ii) and (iii); (ii) and (iv); (ii) and (v); (ii) and (vi); (ii) and (vii); (ii) and (viii); (iii) and (iv); (iii) and (v); (iii) and (vi); (iii) and (vii); (iii) and (viii); (iv) and (v); (iv) and (vi); (iv) and (vii); (iv) and (viii); (v) and (vi); (v) and (vii); (v) and (viii); (vi) and (vii); (vi) and (viii); or (vii) and (viii).

In one embodiment, the process for preparing a compound of Formula (I) comprises at least three of steps (i) to (viii), optionally (i), (ii), and (iii); (i), (ii), and (iv); (i), (ii), and (v); (i), (ii), and (vi); (i), (ii), and (vii); (i), (ii), and (viii); (i), (iii), and (iv); (i), (iii), and (v); (i), (iii), and (vi); (i), (iii), and (vii); (i), (iii), and (viii); (i), (iv), and (v); (i), (iv), and (vi); (i), (iv), and (vii); (i), (iv), and (viii); (i), (v), and (vi); (i), (v), and (vii); (i), (v), and (viii); (i), (vi), and (vii); (i), (vi), and (viii); (i), (vii), and (viii); (ii), (iii), and (iv); (ii), (iii), and (v); (ii), (iii), and (vi); (ii), (iii), and (vii); (ii), (iii), and (viii); (ii), (iv), and (v); (ii), (iv), and (vi); (ii), (iv), and (vii); (ii), (iv), and (viii); (ii), (v), and (vi); (ii), (v), and (vii); (ii), (v), and (viii); (ii), (vi), and (vii); (ii), (vi), and (viii); (ii), (vii), and (viii); (iii), (iv), and (v); (iii), (iv), and (vi); (iii), (iv), and (vii); (iii), (iv), and (viii); (iii), (v), and (vi); (iii), (v), and (vii); (iii), (v), and (viii); (iii), (vi), and (vii); (iii), (vi), and (viii); (iii), (vii), and (viii); (iv), (v), and (vi); (iv), (v), and (vii); (iv), (v), and (viii); (iv), (vi), and (vii); (iv), (vi), and (viii); (iv), (vii), and (viii); (v), (vi), and (vii); (v), (vi), and (viii); (v), (vii), and (viii); or (vi), (vii), and (viii).

In one embodiment, the process for preparing a compound of Formula (I) comprises at least four of steps (i) to (viii), optionally (i), (ii), (iii), and (iv); (i), (ii), (iii), and (v); (i), (ii), (iii), and (vi); (i), (ii), (iii), and (vii); (i), (ii), (iii), and (viii); (i), (ii), (iv), and (v); (i), (ii), (iv), and (vi); (i), (ii), (iv), and (vii); (i), (ii), (iv), and (viii); (i), (ii), (v), and (vi); (i), (ii), (v), and (vii); (i), (ii), (v), and (viii); (i), (ii), (vi), and (vii); (i), (ii), (vi), and (viii); (i), (ii), (vii), and (viii); (i), (iii), (iv), and (v); (i), (iii), (iv), and (vi); (i), (iii), (iv), and (vii); (i), (iii), (iv), and (viii); (i), (iii), (v), and (vi); (i), (iii), (v), and (vii); (i), (iii), (v), and (viii); (i), (iii), (vi), and (vii); (i), (iii), (vi), and (viii); (i), (iii), (vii), and (viii); (i), (iv), (v), and (vi); (i), (iv), (v), and (vii); (i), (iv), (v), and (viii); (i), (iv), (vi), and (vii); (i), (iv), (vi), and (viii); (i), (iv), (vii), and (viii); (i), (v), (vi), and (vii); (i), (v), (vi), and (viii); (i), (v), (vii), and (viii); (i), (vi), (vii), and (viii); (ii), (iii), (iv), and (v); (ii), (iii), (iv), and (vi); (ii), (iii), (iv), and (vii); (ii), (iii), (iv), and (viii); (ii), (iii), (v), and (vi); (ii), (iii), (v), and (vii); (ii), (iii), (v), and (viii); (ii), (iii), (vi), and (vii); (ii), (iii), (vi), and (viii); (ii), (iii), (vii), and (viii); (ii), (iv), (v), and (vi); (ii), (iv), (v), and (vii); (ii), (iv), (v), and (viii); (ii), (iv), (vi), and (vii); (ii), (iv), (vi), and (viii); (ii), (iv), (vii), and (viii); (ii), (v), (vi), and (vii); (ii), (v), (vi), and (viii); (ii), (v), (vii), and (viii); (ii), (vi), (vii), and (viii); (iii), (iv), (v), and (vi); (iii), (iv), (v), and (vii); (iii), (iv), (v), and (viii); (iii), (iv), (vi), and (vii); (iii), (iv), (vi), and (viii); (iii), (iv), (vii), and (viii); (iii), (v), (vi), and (vii); (iii), (v), (vi), and (viii); (iii), (v), (vii), and (viii); (iii), (vi), (vii), and (viii); (iv), (v), (vi), and (vii); (iv), (v), (vi), and (viii); (iv), (v), (vii), and (viii); (iv), (vi), (vii), and (viii); or (v), (vi), (vii), and (viii).

In one embodiment, the process for preparing a compound of Formula (I) comprises at least five of steps (i) to (viii); optionally (i), (ii), (iii), (iv), and (v); (i), (ii), (iii), (iv), and (vi); (i), (ii), (iii), (iv), and (vii); (i), (ii), (iii), (iv), and (viii); (i), (ii), (iii), (v), and (vi); (i), (ii), (iii), (v), and (vii); (i), (ii), (iii), (v), and (viii); (i), (ii), (iii), (vi), and (vii); (i), (ii), (iii), (vi), and (viii); (i), (ii), (iii), (vii), and (viii); (i), (ii), (iv), (v), and (vi); (i), (ii), (iv), (v), and (vii); (i), (ii), (iv), (v), and (viii); (i), (ii), (iv), (vi), and (vii); (i), (ii), (iv), (vi), and (viii); (i), (ii), (iv), (vii), and (viii); (i), (ii), (v), (vi), and (vii); (i), (ii), (v), (vi), and (viii); (i), (ii), (v), (vii), and (viii); (i), (ii), (vi), (vii), and (viii); (i), (iii), (iv), (v), and (vi); (i), (iii), (iv), (v), and (vii); (i), (iii), (iv), (v), and (viii); (i), (iii), (iv), (vi), and (vii); (i), (iii), (iv), (vi), and (viii); (i), (iii), (iv), (vii), and (viii); (i), (iii), (v), (vi), and (vii); (i), (iii), (v), (vi), and (viii); (i), (iii), (v), (vii), and (viii); (i), (iii), (vi), (vii), and (viii); (i), (iv), (v), (vi), and (vii); (i), (iv), (v), (vi), and (viii); (i), (iv), (v), (vii), and (viii); (i), (iv), (vi), (vii), and (viii); (i), (v), (vi), (vii), and (viii); (ii), (iii), (iv), (v), and (vi); (ii), (iii), (iv), (v), and (vii); (ii), (iii), (iv), (v), and (viii); (ii), (iii), (iv), (vi), and (vii); (ii), (iii), (iv), (vi), and (viii); (ii), (iii), (iv), (vii), and (viii); (ii), (iii), (v), (vi), and (vii); (ii), (iii), (v), (vi), and (viii); (ii), (iii), (v), (vii), and (viii); (ii), (iii), (vi), (vii), and (viii); (ii), (iv), (v), (vi), and (vii); (ii), (iv), (v), (vi), and (viii); (ii), (iv), (v), (vii), and (viii); (ii), (iv), (vi), (vii), and (viii); (ii), (v), (vi), (vii), and (viii); (iii), (iv), (v), (vi), and (vii); (iii), (iv), (v), (vi), and (viii); (iii), (iv), (v), (vii), and (viii); (iii), (iv), (vi), (vii), and (viii); (iii), (v), (vi), (vii), and (viii); or (iv), (v), (vi), (vii), and (viii).

In one embodiment, the process for preparing a compound of Formula (I) comprises at least six of steps (i) to (viii), optionally (i), (ii), (iii), (iv), (v), and (vi); (i), (ii), (iii), (iv), (v), and (vii); (i), (ii), (iii), (iv), (v), and (viii); (i), (ii), (iii), (iv), (vi), and (vii); (i), (ii), (iii), (iv), (vi), and (viii); (i), (ii), (iii), (iv), (vii), and (viii); (i), (ii), (iii), (v), (vi), and (vii); (i), (ii), (iii), (v), (vi), and (viii); (i), (ii), (iii), (v), (vii), and (viii); (i), (ii), (iii), (vi), (vii), and (viii); (i), (ii), (iv), (v), (vi), and (vii); (i), (ii), (iv), (v), (vi), and (viii); (i), (ii), (iv), (v), (vii), and (viii); (i), (ii), (iv), (vi), (vii), and (viii); (i), (ii), (v), (vi), (vii), and (viii); (i), (iii), (iv), (v), (vi), and (vii); (i), (iii), (iv), (v), (vi), and (viii); (i), (iii), (iv), (v), (vii), and (viii); (i), (iii), (iv), (vi), (vii), and (viii); (i), (iii), (v), (vi), (vii), and (viii); (i), (iv), (v), (vi), (vii), and (viii); (ii), (iii), (iv), (v), (vi), and (vii); (ii), (iii), (iv), (v), (vi), and (viii); (ii), (iii), (iv), (v), (vii), and (viii); (ii), (iii), (iv), (vi), (vii), and (viii); (ii), (iii), (v), (vi), (vii), and (viii); (ii), (iv), (v), (vi), (vii), and (viii); or (iii), (iv), (v), (vi), (vii), and (viii).

In one embodiment, the process for preparing a compound of Formula (I) comprises at least seven of steps (i) to (viii); optionally (i), (ii), (iii), (iv), (v), (vi), and (vii); (i), (ii), (iii), (iv), (v), (vi), and (viii); (i), (ii), (iii), (iv), (v), (vii), and (viii); (i), (ii), (iii), (iv), (vi), (vii), and (viii); (i), (ii), (iii), (v), (vi), (vii), and (viii); (i), (ii), (iv), (v), (vi), (vii), and (viii); (i), (iii), (iv), (v), (vi), (vii), and (viii); or (ii), (iii), (iv), (v), (vi), (vii), and (viii).

In one embodiment, compound (Ib) is telescoped to the next step in its crude form (i.e. without isolation and/or purification). In another embodiment, compound (If) is telescoped to the next step in its crude form (i.e. without isolation and/or purification).

More specifically, the synthetic route for the synthesis of the compound of formula (I) is described below in Scheme 2. The square brackets indicate compounds that may be telescoped through to the next step in their crude form (i.e. without isolation and/or purification). Compound (Ia1) is pictured as the hydrochloride salt, but the corresponding freebase or a different salt thereof may be used.

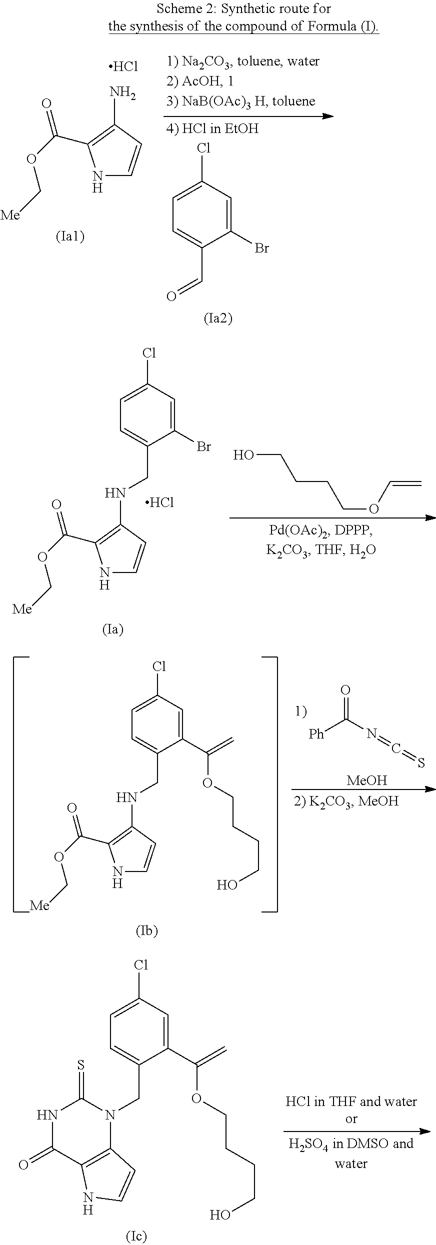

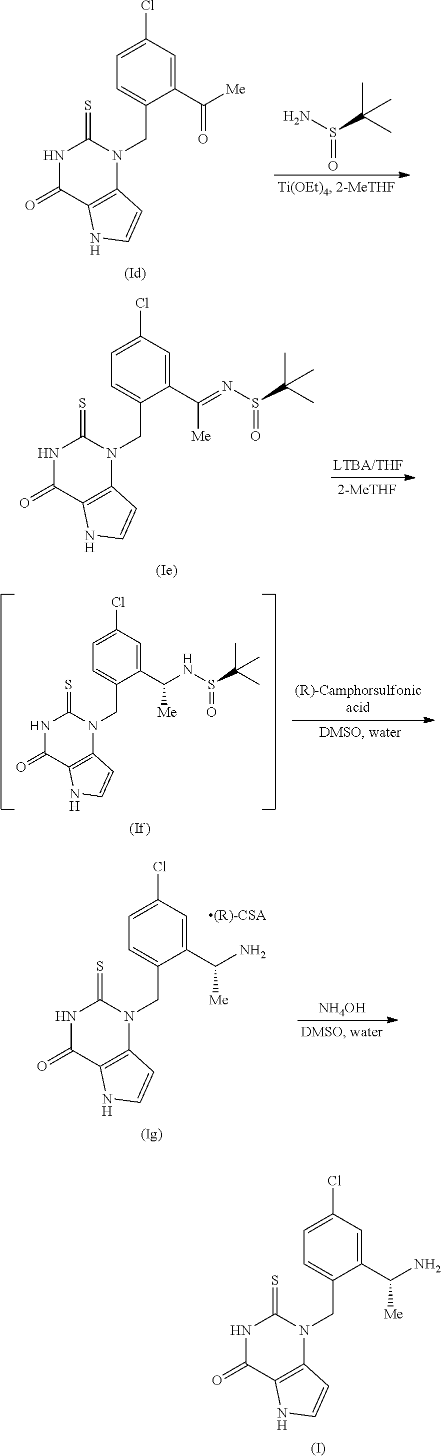

In one embodiment, step (i) comprises at least the following steps;

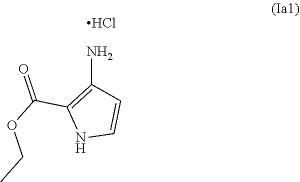

(i-a) breaking the hydrochloride salt of a compound of Formula (Ia1), or the corresponding freebase or a different salt thereof, in the presence of an inorganic base, optionally wherein the inorganic base is sodium carbonate;

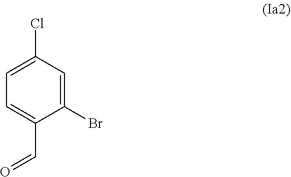

(i-b) reacting the resulting compound of step (i-a) with a compound of Formula (Ia2) under acidic conditions, optionally wherein the acid is acetic acid;
(i-c) reducing the resulting imine compound of step (i-b) by reacting with a reducing agent, optionally wherein the reducing reagent is sodium triacetoxyborohydride; and
(i-d) crystallising the resulting compound of Formula (Ia) from step (c) as the hydrochloride salt.

In one embodiment, (ii) comprises at least (ii-a) reacting a compound of Formula (Ia) with 4-(vinyloxy)butan-1-ol in the presence of a palladium catalyst, optionally wherein the palladium catalyst is palladium acetate, optionally also in the presence of a phosphine ligand, preferably wherein the phosphine ligand is one of 1,3-bis(diphenylphosphino)propane (DPPP), 2-dicyclohexylphosphin-2′, 4′,6′-triisopropylbiphenyl (X-Phos) or 1,3-bis(diphenylphosphino)benzene.

In one embodiment, step (iii) comprises at least (iii-a) reacting a compound of Formula (Ib) with benzoyl isothiocyanate.

In one embodiment, step (iv) comprises at least (iv-a) reacting a compound of Formula (Ic) with a inorganic acid, optionally wherein the inorganic acid is hydrochloric acid or sulfuric acid.

In one embodiment, step (v) comprises at least (v-a) reacting a compound of Formula (Id) with a chiral sulfinamide reagent in the presence of a dehydrating reagent, optionally wherein the dehydrating reagent is titanium ethoxide.

In one embodiment, step (vi) comprises at least (vi-a) reacting a compound of Formula (Ie) with a reducing agent, optionally wherein the reducing agent is lithium tri-tert-butoxyaluminum hydride.

In one embodiment, step (vii) comprises at least (vii-a) reacting a compound of Formula (If) with a chiral resolving agent, optionally wherein the compound of Formula (If) is provided from preceding step (vi) in an organic solvent without purification.

In one embodiment, step (viii) comprises at least (viii-a) reacting a compound of Formula (Ig) with an inorganic base to provide a compound of Formula (I).

In one embodiment the base is an inorganic base selected from a hydroxide, carbonate or bicarbonate salt. In one embodiment the base is an inorganic base that is a non-metal hydroxide. In one embodiment the base is ammonium hydroxide.

In a second aspect there is provided a compound selected from the group consisting of:

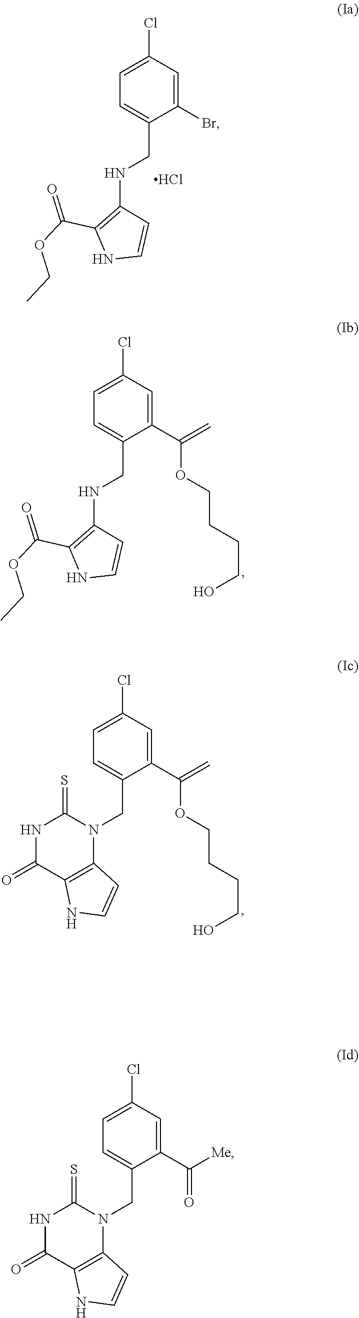 [SMILES: CCOC(=O)c1[nH]ccc1NCc1ccc(Cl)cc1Br ; Cl ; C=C(OCCCCO)c1cc(Cl)ccc1CNc1cc[nH]c1C(=O)OCC ; C=C(OCCCCO)c1cc(Cl)ccc1Cn1c(=S)[nH]c(=O)c2[nH]ccc21 ; CC(=O)c1cc(Cl)ccc1Cn1c(=S)[nH]c(=O)c2[nH]ccc21]

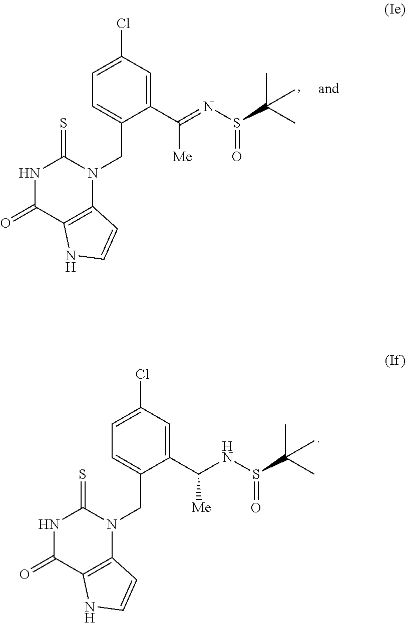 [SMILES: C/C(=N\[S@](=O)C(C)(C)C)c1cc(Cl)ccc1Cn1c(=S)[nH]c(=O)c2[nH]ccc21 ; C[C@@H](N[S@](=O)C(C)(C)C)c1cc(Cl)ccc1Cn1c(=S)[nH]c(=O)c2[nH]ccc21]

In a third aspect there is provided a compound having the structure:

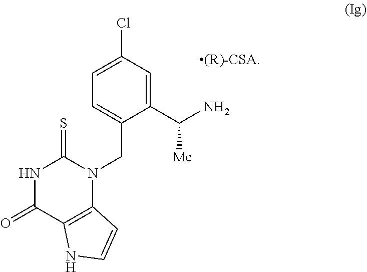 [SMILES: C[C@@H](N)c1cc(Cl)ccc1Cn1c(=S)[nH]c(=O)c2[nH]ccc21]

### DETAILED DESCRIPTION

### Stage 1/Step (i): Reductive Amination

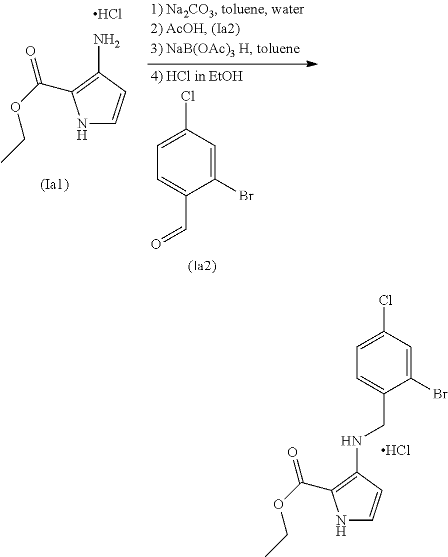 [SMILES: Cl ; CCOC(=O)c1[nH]ccc1N ; O=Cc1ccc(Cl)cc1Br ; CCOC(=O)c1[nH]ccc1NCc1ccc(Cl)cc1Br ; Cl]

The salt break step may be carried out in a mixture of water with a variety of organic solvents such as dichloromethane, 2-methyltetrahydrofuran, isopropylacetate or toluene. In one aspect, the salt break was carried out in water and toluene. The salt break may be performed using a variety of bases such as sodium carbonate, potassium carbonate or caesium carbonate. In one aspect, sodium carbonate was used as base.

The reaction may be carried out in a variety of organic solvents such as dichloromethane, ethanol, methanol, tetrahydrofuran, isopropylalcohol, dioxane and toluene. In one aspect, the reaction was carried out in toluene. A variety of acids may be used, such as trifluoroacetic acid, hydrochloric acid, toluenesulfonic acid or acetic acid. Alternatively, the reaction may be carried out in the absence of acid. In one aspect, acetic acid was used as acid. The reduction stage may be carried out by methods which will be familiar to those skilled in the art. A variety of reducing agents such as lithium aluminium hydride, sodium cyanoborohydride, sodium borohydride, palladium on carbon or sodium triacetoxyborohydride may be used. In one aspect, sodium triacetoxyborohydride was used for the reduction step.

The reaction may be carried out at a range of temperatures, for example −40° C. to 30° C. In one aspect, the reaction was carried out between −10° C. to 10° C. In one aspect, ranges disclosed herein are inclusive of the stated endpoints (e.g. the range between −10° C. to 10° C. includes −10° C. and 10° C.).

The product may be crystallised as the HCl salt by addition of hydrochloric acid in methanol, ethanol, or isopropyl alcohol. In one aspect, hydrochloric acid in ethanol was used.

### Stage 2A/Step (ii): Mizoroki-Heck Coupling

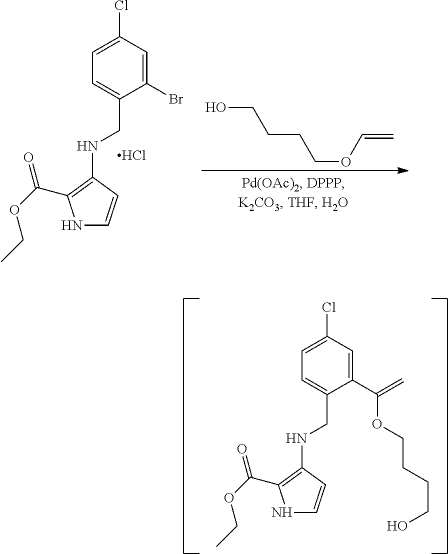

The reaction may be carried out in a variety of solvents or mixtures of solvents, including water, dioxane, methanol, ethanol, tetrahydrofuran, 2-methyltetrahydrofuran, toluene and isopropylalcohol. In one aspect, a mixture of polar solvents is preferred. In another aspect, a mixture of a polar aprotic and polar protic solvent is preferred. In one aspect, the solvent comprises a mixture of tetrahydrofuran and water. The reaction may be carried out using a variety of bases such as potassium carbonate, sodium carbonate, triethylamine or sodium hydroxide. In one aspect, potassium carbonate was used as a base. The reaction may be carried out using a variety of palladium catalysts, such as tetrakis(triphenylphosphine)palladium(0), palladium acetate, [1,1′-bis(diphenylphosphino)ferrocene]palladium(II) dichloride or dichloro bis(triphenylphosphine)palladium(II), or any other catalyst which will be familiar to those skilled in the art. In one aspect, palladium acetate was used as the catalyst. A range of ligands may also be used, such as 1,3-bis(diphenylphosphino)propane (DPPP), X-Phos or 1,3-bis(diphenylphosphino)benzene. In one aspect, 1,3-bis(diphenylphosphino)propane was used as the ligand.

The reaction may be carried out at a range of temperatures, for example 40° C. to 150° C. In one aspect, the reaction was carried out between 70° C. and 85° C.

Stage 2B/Step (iii): Cyclisation

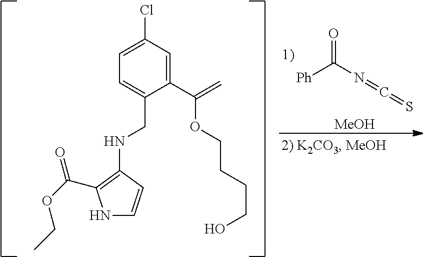

The reaction may be carried out in a variety of organic solvents such as toluene, methanol, ethanol, ethyl acetate or tetrahydrofuran. In one aspect, a polar solvent is preferred. In another aspect, a polar protic solvent is preferred. In one aspect, the reaction was carried out in methanol. The cyclisation step may be carried out using a variety of bases such as potassium carbonate, sodium carbonate, cesium carbonate, potassium hydroxide, sodium hydroxide or sodium methoxide. In one aspect inorganic bases are preferred. In another aspect, carbonates, hydroxides or alkoxides are preferred. In one aspect, potassium carbonate was used as the base.

The reaction may be carried out at a range of temperatures, for example 0° C. to 70° C. In one aspect, the first step of the reaction was carried out between 0° C. and 10° C. and the cyclisation step between 40° C. and 60° C.

### Stage 3/Step (iv): Enol Ether Cleavage

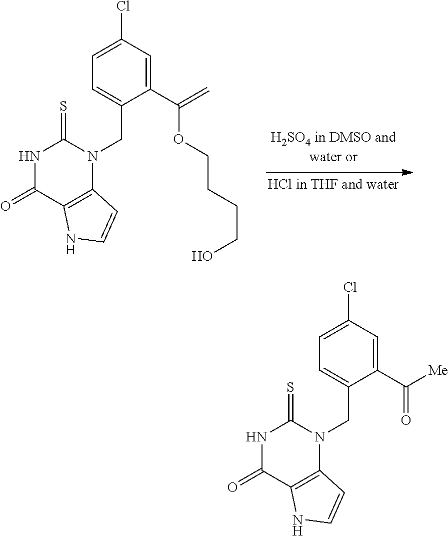 [SMILES: C=C(OCCCCO)c1cc(Cl)ccc1Cn1c(=S)[nH]c(=O)c2[nH]ccc21 ; CC(=O)c1cc(Cl)ccc1Cn1c(=S)[nH]c(=O)c2[nH]ccc21]

The reaction may be carried out in a variety of solvents including water, tetrahydrofuran, 2-methyltetrahydrofuran, methanol, ethanol, dimethylsulfoxide or a mixture of solvents. In one aspect, a polar solvent is preferred. In another aspect, a polar aprotic solvent is preferred. In one aspect, tetrahydrofuran was used. A number of acids may be used, for example hydrochloric acid, sulfuric acid, trifluoroacetic acid or methylsulfonic acid. In one aspect, inorganic acids are preferred. In one aspect, hydrochloric acid was used. In one aspect sulfuric acid was used.

The reaction may be carried out at a range of temperatures, for example 0° C. to 50° C. In one aspect, the reaction was carried out between 10° C. and 30° C.

### Stage 4/Step (v): Sulfinimine Formation

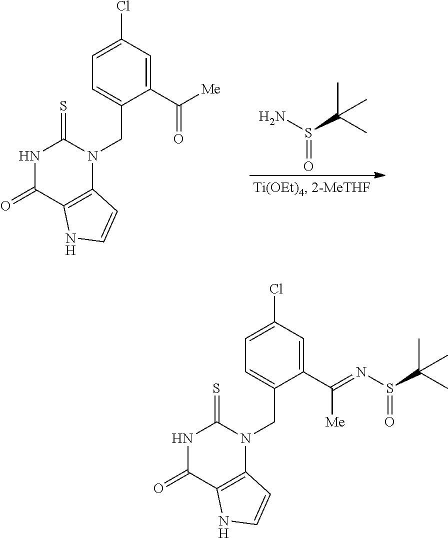

The reaction may be carried out in a variety of organic solvents such as tetrahydrofuran, 2-methyltetrahydrofuran, dioxane, acetonitrile, isopropyl alcohol or ethyl acetate. In one aspect, a polar solvent is preferred. In another aspect, a polar aprotic solvent is preferred. In one aspect, 2-methyltetrahydrofuran was used as the reaction solvent. The reaction may be carried out using a variety of dehydrating agents which will be familiar to those skilled in the art such as titanium ethoxide, titanium isopropoxide or titanium chloride. In one aspect, titanium ethoxide is used for this reaction. In another aspect, (R)- or (S)-tert-butanesulfinamide may be used, preferably (R)-tert-butanesulfinamide. The reaction may be carried out at a range of temperatures, for example 40° C. to 100° C. In one aspect, the reaction was carried out between 70° C. and 90° C.

### Stage 5/Step (vi): Stereoselective Reduction

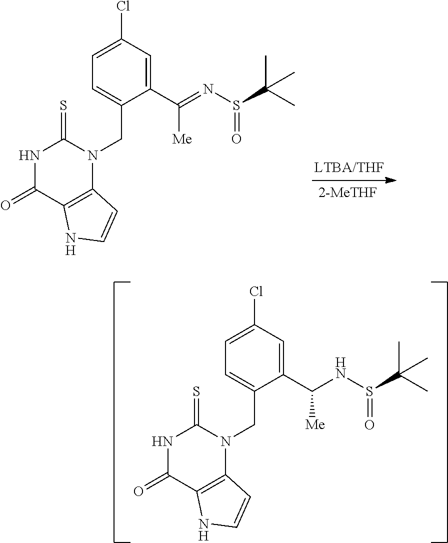

The reaction may be carried out in a variety of organic solvents such as tetrahydrofuran, 2-methyltetrahydrofuran, tent-butyl methyl ether, toluene or a mixture of solvents. In one aspect, polar solvents are preferred. In another aspect, a mixture of polar aprotic solvents are preferred. In one aspect, a mixture of 2-methyletrahydrofuran and tetrahydrofuran were used for the reaction. The reaction may be carried out using a variety of reducing agents which will be familiar to those skilled in the art such as L-Selectride®, diisobutylaluminium hydride, lithium aluminium hydride, sodium borohydride or lithium tri-tert-butoxyylaluminium hydride (LTBA). In one aspect, lithium tri-tert-butoxyylaluminium hydride (LTBA) was used as the reducing agent with (R)-N-[(1E)-1-[5-chloro-2-[(4-oxo-2-sulfanylidene-2,3,4,5-tetrahydro-1H-pyrrolo[3,2-d]pyrimidin-1-yl)methyl]phenyl]ethylidene]-2-methylpropane-2-sulfinamide. In one aspect, L-Selectride® is used as the reducing agent with the (S)-N-[(1E)-1-[5-chloro-2-[(4-oxo-2-sulfanylidene-2,3,4,5-tetrahydro-1H-pyrrolo[3,2-d]pyrimidin-1-yl)methyl]phenyl]ethylidene]-2-methylpropane-2-sulfinamide. The reaction may be carried out at a range of temperatures, for example—40° C. to 50° C. In one aspect, the reaction was carried out between 0° C. and 20° C. The product may be crystallised or telescoped through to the next stage as a solution in organic solvent. In one aspect, the product was crystallised from methanol. In one aspect, the product is subsequently telescoped through to the next stage as a solution in DMSO.

Stage 6/Step (vii): Sulfinyl Group Cleavage

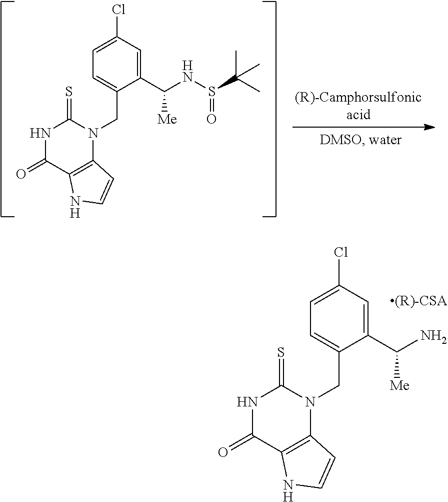

The reaction may be carried out in a range of organic solvents such as water, ethanol, methanol, tetrahydrofuran, 2-methyltetrahydrofuran, dimethylsulfoxide or a mixture of solvents. In one aspect, polar solvents are preferred. In another aspect, a mixture of a polar aprotic and polar protic solvent is preferred. In one aspect, the reaction was carried out in a mixture of dimethylsulfoxide and water. In another aspect, (R)- or (S)-camphorsulfonic acid may be used, preferably having the same stereoconfiguration as the chiral amine. The reaction may be carried out at a range of temperatures, for example 20° C. to 80° C. In one aspect, the reaction was carried out between 40° C. and 70° C.

Stage 7/Step (viii): Freebase Formation

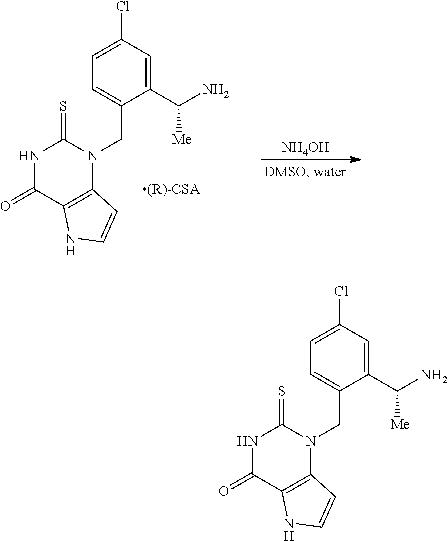 [SMILES: C[C@@H](N)c1cc(Cl)ccc1Cn1c(=S)[nH]c(=O)c2[nH]ccc21 ; "NH4OH" ; C[C@@H](N)c1cc(Cl)ccc1Cn1c(=S)[nH]c(=O)c2[nH]ccc21]

The reaction may be carried out in a range of solvents such as water, ethanol, methanol, tetrahydrofuran, 2-methyltetrahydrofuran, dimethylsulfoxide or a mixture of solvents. In one aspect, a mixture of polar solvents is preferred. In another aspect, a mixture of a polar aprotic and a polar protic solvent is preferred. In one aspect, the reaction was carried out in dimethylsulfoxide and water. The reaction may be carried out using a variety of bases such as ammonium hydroxide, triethylamine and diisopropylamine. In one aspect, ammonium hydroxide was used as base.

The reaction may be carried out at a range of temperatures, for example 10° C. to 60° C. In one aspect, the reaction was carried out between 15° C. and 45° C.

### EXAMPLES

### General Methods

Unless stated otherwise, starting materials were commercially available. All solvents and commercial reagents were of laboratory grade and were used as received. Unless stated otherwise, all operations were carried out at ambient temperature, i.e. in the range 17 to 28° C. and, where appropriate, under an atmosphere of an inert gas such as nitrogen.

Large scale reactions were carried out in stainless steel or glass-lined steel reactors fitted with heat transfer jackets and serviced with appropriate ancillary equipment.

When given, ¹H NMR spectra were recorded on a Bruker A500 (500 MHz), a Bruker A400 (400 MHz). Either the central peaks of chloroform-d (CDCl₃; δH 7.27 ppm) or dimethylsulfoxide-d6 (d6-DMSO; δH 2.50 ppm), or an internal standard of tetramethylsilane (TMS; δH 0.00 ppm) were used as references. Sample solutions may also contain an internal standard (for example maleic acid or 2,3,5,6-tetrachloronitrobenzene) for assay determination. Spectral data is reported as a list of chemical shifts (δ, in ppm) with a description of each signal, using standard abbreviations (s=singlet, d=doublet, m=multiplet, t=triplet, q=quartet, br=broad, etc.). It is well known in the art that chemical shifts and J-coupling constants may vary slightly as a result of sample preparation differences, for example analyte concentration and whether or not additives (for example NMR assay standards) are included.

It is to be understood that each of the reaction parameters in all examples disclosed herein (e.g. temperatures, times, reagent ratios and/or amounts, workups, purifications) are also individually and generally disclosed.

### Stage 1/Step (i): Reductive Amination

Ethyl 3-[[(2-bromo-4-chlorophenyl)methyl]amino]-1H-pyrrole-2-carboxylate hydrochloride

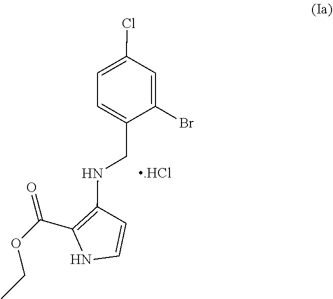 [SMILES: CCOC(=O)c1[nH]ccc1NCc1ccc(Cl)cc1Br ; "·.HCl"]

Ethyl 3-amino-1H-pyrrole-2-carboxylate hydrochloride (110 kg, 577 mol, 1.0 eq.) and toluene (2200 L, 20 vols) were charged to the vessel at 25° C. Water (550 L, 5 vols) and sodium carbonate (61.6 kg, 577 mol, 1.0 eq.) were charged and the contents of the vessel were stirred for 10 minutes until a biphasic solution was formed. The batch was allowed to settle for 30 minutes before the aqueous phase was removed. Sodium chloride (55 kg, 50% w/w) was charged into the aqueous phase and it was back extracted with toluene (550 L, 5 vols). The combined organic phases were cooled to 0° C. and acetic acid (86.9 kg, 1442 mol, 2.5 eq.) and 2-bromo-4-chlorobenzaldehyde (151.8 kg, 692 mol, 1.2 eq.) were charged. The mixture was held at 0° C. for 20 minutes. Sodium triacetoxyborohydride (183.7 kg, 865 mol, 1.5 eq.) was charged, keeping the temperature below 5° C., and the mixture was held for 20 minutes at 0° C. Aqueous ammonia solution (242 L 25-28% ammonia solution in 770 L water) was charged to the vessel at 15° C. The mixture was held for 1 hour. The batch was allowed to settle and the aqueous phase was removed. The aqueous phase was back extracted with toluene (110 L, 1 vol) and the combined organic phases were washed twice with water (2×550 L, 5 vols). A solution of anhydrous HCl in ethanol (89.1 kg, 807 mol, 1.4 eq.) was charged at 20° C. and the mixture held for 30 minutes. The resulting solid was collected by filtration. The filter cake was washed with toluene (220 L, 2 vols) and dried at 55° C. to yield ethyl 3-[[(2-bromo-4-chlorophenyl)methyl]amino]-1H-pyrrole-2-carboxylate hydrochloride (205.7 kg, 86.5% w/w free base assay, 519 mol, 90% yield) as a solid.

¹H NMR (400 MHz, DMSO, 27° C.) 1.28 (3H, t), 4.21 (2H, q), 4.32 (2H, d), 5.96 (1H, s), 6.71 (1H, t), 7.32-7.45 (2H, m), 7.47-7.78 (2H, m), 10.81 (1H, s).

### Stage 2A/Step (ii): Mizoroki—Heck Coupling

Ethyl 3-[([4-chloro-2-[1-(4-Hydroxybutoxy)ethenyl]phenyl]methyl)amino]-1H-pyrrole-2-carboxylate

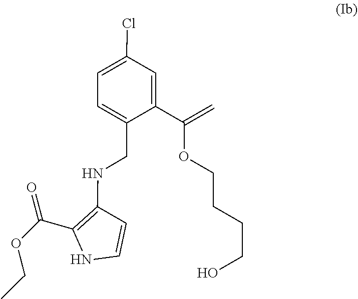 [SMILES: C=C(OCCCCO)c1cc(Cl)ccc1CNc1cc[nH]c1C(=O)OCC]

Ethyl 3-[[(2-bromo-4-chlorophenyl)methyl]amino]-1H-pyrrole-2-carboxylate hydrochloride (205 kg, 520 mol, 1.0 eq), tetrahydrofuran (472 L, 2.3 vols), water (1578 L, 7.7 vols) and potassium carbonate (215.3 kg, 1560 mol, 3.0 eq.) were charged to the vessel. 4-(Vinyloxy)butan-1-ol (180.4 kg, 1560 mol, 3.0 eq), 1,3-bis(diphenylphosphino)propane (6.56 kg, 15.6 mol, 0.03 eq) and palladium (II) acetate (1.74 kg, 7.8 mol, 0.015 eq) were charged, the solution was degassed using nitrogen and the reaction mixture was heated to 75° C. for 20 hours. The mixture was cooled to approximately 25° C. and ethyl acetate (1025 L, 5 vols) was charged. Acetyl cysteine (127.1 kg, 780 mol, 1.5 eq) was charged and the resulting biphasic solution stirred at 25° C. for 3 hours. The batch was allowed to settle and the aqueous phase was removed. The aqueous phase was back extracted with ethyl acetate (1025 L, 5 vols) and the combined organic phases were washed twice with aqueous sodium chloride solution (2×1025 L, 5 vols). The resulting organic phase was distilled to approximately 2.5 volumes under reduced pressure. Methanol (1025 L, 5 vols) was charged and the batch was distilled to approximately 2.5 volumes under reduced pressure to yield ethyl 3-[([4-chloro-2-[1-(4-Hydroxybutoxy)ethenyl]phenyl]methyl)amino]-1H-pyrrole-2-carboxylate (705.3 kg, 29.5% w/w assay, quantitative yield).

¹H NMR (400 MHz, CDCl₃, 27° C.) 1.32 (3H, t), 1.65-1.70 (3H, m), 1.77-1.86 (2H, m), 3.66 (2H, t), 3.87 (2H, t), 4.17-4.32 (3H, m), 4.33-4.40 (3H, m), 5.63 (1H, t), 6.67 (1H, s), 7.18-7.29 (1H, m), 7.3-7.42 (2H, m), 7.73 (2H, s).

Stage 2B/Step (iii): Cyclisation

1-([4-Chloro-2-[1-(4-hydroxybutoxy)ethenyl]phenyl]methyl)-2-sulfanylidene-1,2,3,5-tetrahydro-4H-pyrrolo[3,2-d]pyrimidin-4-one

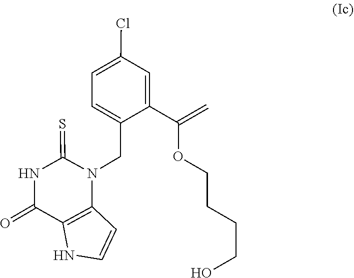 [SMILES: C=C(OCCCCO)c1cc(Cl)ccc1Cn1c(=S)[nH]c(=O)c2[nH]ccc21]

A solution of ethyl 3-[([4-chloro-2-[1-(4-Hydroxybutoxy)ethenyl]phenyl]methyl)amino]-1H-pyrrole-2-carboxylate in methanol (705 kg, 29.5% w/w assay, 496 mol, 1.0 eq) was charged to the vessel. Methanol (585 L, 3 vols) was charged and the resulting solution was cooled to 5° C. Benzoyl isothiocyanate (93.6 kg, 571 mol, 1.15 eq.) was charged dropwise, keeping the temperature of the batch below 10° C. The reaction was held for 1 hour. Potassium carbonate (150.2 kg, 1092 mol, 2.2 eq.) was charged and the mixture was heated to 45° C. and held for 8 hours. The reaction was cooled to 25° C. and acetic acid (134.6 kg, 2233 mol, 4.5 eq.) was charged dropwise over 1.5 hours. The mixture was held for 30 minutes. Ethyl acetate (1560 L, 8 vols) and water (975 L, 5 vols) were charged and the biphasic mixture was held for 15 minutes. The batch was allowed to settle and the aqueous phase was removed. The aqueous phase was back extracted twice with ethyl acetate (585 L, 3 vols, then 390 L, 2 vols). The combined organic phases were distilled to approximately 5 volumes under reduced pressure. Ethyl acetate (975 L, 5 vols) was charged and the batch was distilled to approximately 5 vols. Ethyl acetate (975 L, 5 vols) was charged and the batch was distilled to approximately 5 vols. The resulting slurry was cooled to 5° C. and held for 5 minutes. The solids were collected by filtration and the filter cake was washed with ethyl acetate (780 L, 4 vols) which had been cooled to 0° C. The solids were dried at 50° C. to yield 1-([4-chloro-2-[1-(4-hydroxybutoxy)ethenyl]phenyl]methyl)-2-sulfanylidene-1,2,3,5-tetrahydro-4H-pyrrolo[3,2-d]pyrimidin-4-one (195 kg, 95.7% w/w, 76% yield) as a solid.

¹H NMR (500 MHz, DMSO, 27° C.) 1.50-1.59 (2H, m), 1.70-1.80 (2H, m), 3.43 (2H, t), 3.89 (2H, t), 4.43 (1H, d), 4.56 (1H, d), 5.64-5.68 (2H, m), 5.77 (1H, d), 6.88 (1H, d), 7.23 (1H, d), 7.31 (1H, dd), 7.38 (1H, d). Exchangeable protons not seen.

### Stage 3/Step (iv): Enol Ether Cleavage

1-[(2-Acetyl-4-chlorophenyl)methyl]-2-sulfanylidene-1,2,3,5-tetrahydro-4H-pyrrolo[3,2-d]pyrimidin-4-one

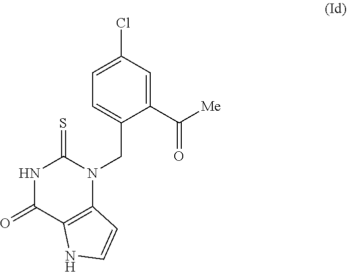 [SMILES: CC(=O)c1cc(Cl)ccc1Cn1c(=S)[nH]c(=O)c2[nH]ccc21]

Method a: 1-([4-chloro-2-[1-(4-hydroxybutoxy)ethenyl]phenyl]methyl)-2-sulfanylidene -1,2,3,5-tetrahydro-4H-pyrrolo[3,2-d]pyrimidin-4-one (236 kg, 560 mol, 1.0 eq.) and tetrahydrofuran (1888 L, 8 vols) were charged to the vessel. Concentrated hydrochloric acid (59 kg, 560 mol, 1.0 eq) was charged dropwise, maintaining the temperature of the batch at 25° C. The reaction was held for 1 hour. The resulting slurry was cooled to 10° C. and water (2832 L, 12 vols) was charged dropwise. The batch was cooled to 5° C. and held for 1 hour before the solids were isolated by filtration. The filter cake was washed twice with water (2×472 L, 2 vols) and dried at 60° C. to yield 1-[(2-acetyl-4-chlorophenyl)methyl]-2-sulfanylidene-1,2,3,5-tetrahydro-4H-pyrrolo[3,2-d]pyrimidin-4-one (237 kg, 543 mol, 97% yield) as a solid.

¹H NMR (500 MHz, DMSO, 27° C.) 2.71 (3H, s), 5.81 (2H, s), 6.09 (1H, d), 6.86 (1H, d), 7.31 (1H, d), 7.51 (1H, dd), 8.07 (1H, d), 12.36 (1H, s), 12.49 (1H, s).

Alternative method b: 1-([4-chloro-2-[1-(4-hydroxybutoxy)ethenyl]phenyl]methyl)-2-sulfanylidene-1,2,3,5-tetrahydro-4H-pyrrolo[3,2-d]pyrimidin-4-one (76 kg, 187 mol, 1.0 eq.) and dimethyl sulfoxide (577.6, 7.6 vols) were charged to the vessel. The mixture was stirred for 30 minutes at 25° C. and was screened. Concentrated sulfuric acid (14.4 kg, 0.75 eq) in water (47.9 L, 0.63 vol) was charged. The mixture was heated to 40° C. and held for 2 hours. Methanol (152 L, 2 vol) was added over 1.5 hours. 1-(2-acetyl-4-chlorobenzyl)-2-sulfanylidene-1,2,3,5 -tetrahydro-4H-pyrrolo [3,2-d]pyrimidin-4-one seed (0.76 kg, 0.01 wt) was added, followed by 3:2 mixture of aqueous methanol (380 L, 5 vol) over 3 hours. The mixture was held at 40° C. for 4 hours and water (152 L, 2 vol) was added over 1 hour. The mixture is cooled to 20° C. over 5 hours and was held at 20° C. for 1 hour, before the solids were isolated by filtration. The filter cake was washed twice with water (2×304 L, 4 vols) and dried at 50° C. to yield 1-[(2-acetyl-4-chlorophenyl)methyl]-2-sulfanylidene-1,2,3,5 - tetrahydro-4H-pyrrolo[3,2-d]pyrimidin-4-one (61.04 kg, 98.6% w/w, 96% yield) as a solid. The seeding in stage 3 was to ensure that the solid was more easily isolatable by filtration on a large scale and to improve manufacturability. In an alternative method that was performed, the steps were identical, but a seed was not used and this did not impact overall yield. The seed used in the method above was made using the stage 3 method a on a scale of 0.4 kg.

### Stage 4/Step (v): Sulfinimine Formation

(R)-N-[(1E)-1-[5-chloro-2-[(4-oxo-2-sulfanylidene-2,3,4,5-tetrahydro-1H-pyrrolo[3,2-d]pyrimidin-1-yl)methyl]phenyl]ethylidene]-2-methylpropane-2-sulfinamide

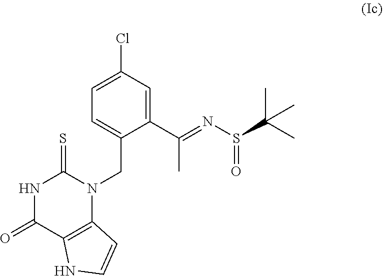 [SMILES: C/C(=N\[S@](=O)C(C)(C)C)c1cc(Cl)ccc1Cn1c(=S)[nH]c(=O)c2[nH]ccc21]

1-[(2-Acetyl-4-chlorophenyl)methyl]-2-sulfanylidene-1,2,3,5-tetrahydro-4H-pyrrolo[3,2-d]pyrimidin-4-one (115 kg, 344 mol, 1.0 eq), 2-methyltetrahydrofuran (575 L, 5 vols), (R)-(+)-2-methyl-2-propanesulfinamide (62 kg, 502 mol, 1.5 eq) and titanium ethoxide (236 kg, 1004 mol, 3.0 eq) were charged to the vessel. The resulting slurry was heated to 80° C. and held for 20 hours. The batch was cooled to 25° C. and 2-methyltetrahydrofuran (1725 L, 15 vols) was charged. Aqueous potassium glycolate solution (5 M, 15 vols) and tetrahydrofuran (690 L, 6 vols) were charged and the resulting biphasic solution was held for 1 hour. The batch was allowed to settle and the aqueous phase was removed. The aqueous phase was back extracted with 2-methyltetrahydrofuran (575 L, 5 vols) and the combined organic phases were washed with 5% aqueous sodium bicarbonate solution (575 L, 5 vols) followed by 15% aqueous sodium chloride solution (575 L, 5 vols). The contents of the vessel were distilled to 3.5 vols under reduced pressure. Methanol (575 L, 5 vols) was charged. The contents of the vessel were distilled to 3.5 vols under reduced pressure and further methanol (345 L, 3 vols) was charged. The resulting slurry was cooled to 0° C. and held for 30 minutes. The solids were collected by filtration and washed with pre cooled methanol (230 L, 2 vols). The product was dried at 50° C. to yield (R)-N-[(1E)-1-[5-chloro-2-[(4-oxo-2-sulfanylidene-2,3,4,5-tetrahydro-1H-pyrrolo [3,2-d]pyrimidin-1-yl)methyl]phenyl]ethylidene]-2-methylpropane-2-sulfinamide (115 kg, 98% w/w, 90.5% yield) as a solid.

¹H NMR (400 MHz, DMSO) 1.22 (9H, s), 2.80 (3H, s), 5.62-5.91 (2H, m), 6.09 (1H, d), 6.85 (1H, d), 7.31-7.41 (2H, m), 7.76 (1H, d), 12.37 (1H, s), 12.49 (1H, s).

### Stage 5/Step (vi): Stereoselective Reduction

(R)-N-[(1R)-1-[5-chloro-2-[(4-oxo-2-sulfanylidene-2,3,4,5-tetrahydro-1H-pyrrolo[3,2-d]pyrimidin-1-yl)methyl]phenyl]ethyl]-2-methylpropane-2-sulfinamide

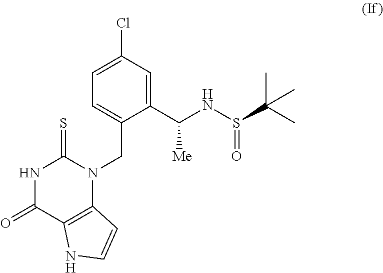 [SMILES: C[C@@H](N[S@](=O)C(C)(C)C)c1cc(Cl)ccc1Cn1c(=S)[nH]c(=O)c2[nH]ccc21]

(R)-N-[(1E)-1-[5-chloro-2-[(4-oxo-2-sulfanylidene-2,3,4,5 -tetrahydro-1H-pyrrolo[3,2-d]pyrimidin-1-yl)methyl]phenyl]ethylidene]-2-methylpropane-2-sulfinamide (127 kg, 292 mol, 1.0 eq.) and 2-methyltetrahydrofuran (1274 L, 10 vols) were charged to the vessel and cooled to 10° C. Lithium tri-tert-butoxyaluminum hydride (496 L, 1 M solution in tetrahydrofuran, 496 mol, 1.7 eq.) was charged dropwise and the resulting mixture was held at 10° C. for 3 hours. A solution of approx. 19% aqueous sodium bisulfate (1274 L, 10 vols) was charged dropwise and the batch was warmed to 25° C. The batch was allowed to separate and the aqueous phase was removed. The organic phase was washed with aqueous sodium chloride solution (637 L, 5 vols) followed by 1 M pH 7.5 phosphate buffer solution (1274 L, 10 vols). 2-Methyltetrahydrofuran (1274 L, 10 vols) was charged and the contents of the vessel were distilled to 8 vols under reduced pressure. 2-Methyltetrahydrofuran (1274 L, 10 vols) was charged and the contents of the vessel were distilled to 8 vols under reduced pressure. The batch was cooled to 25° C. and seeded with (R)-N-[(1R)-1-[5-chloro-2-[(4-oxo-2-sulfanylidene-2,3,4,5-tetrahydro-1H-pyrrolo[3,2-d]pyrimidin-1-yl)methyl]phenyl]ethyl]-2-methylpropane-2-sulfinamide (0.64 kg, 0.5% w/w). The seed was held for 1 hour. Heptane (255 L, 2 vols) was charged over 1 hour and the batch was held for 2 hours. Heptane (382 L, 3 vols) was charged over 1 hour and the batch was held for 2 hours. Heptane (382 L, 3 vols) was charged over 1 hour and the batch was held for 2 hours. Heptane (892 L, 7 vols) was charged over 1 hour and the batch was held for 8 hours. The slurry was filtered and the filter cake was washed twice with heptane (2×64 L, 0.5 vols). The product was dried at 50° C. to yield (R)-N-[(1R)-1-[5-chloro-2-[(4-oxo-2-sulfanylidene-2,3,4,5 -tetrahydro-1H-pyrrolo[3,2-d]pyrimidin-1-yl)methyl]phenyl]ethyl]-2-methylpropane-2-sulfinamide (143 kg, 82.8% assay, 92% yield) as a solid. The seeding in stage 5 was to ensure that the solid was more easily isolatable by filtration on a large scale and to improve manufacturability. In an alternative method that was performed, the steps were identical, but a seed was not used and this did not impact overall yield. The seed used in the method above was made using the alternative method on a scale of 0.05 kg.

¹H NMR (500 MHz, DMSO, 27° C.) 1.14 (9H, s), 1.47 (3H, d), 4.70-4.78 (1H, m), 5.65 (1H, d), 5.81 (1H, d), 5.83 (1H, d), 6.03 (1H, d), 6.61 (1H, d), 7.16 (1H, dd), 7.31 (1H, d), 7.60 (1H, d), 12.07-12.79 (2H, br m).

Stage 6/Step (vii): Sulfinyl Group Cleavage and Chiral Resolution

[(1R)-1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate

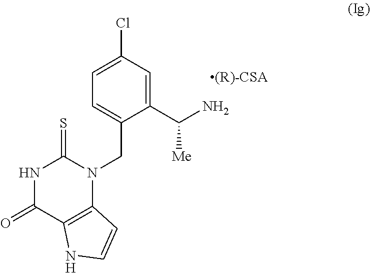 [SMILES: C[C@@H](N)c1cc(Cl)ccc1Cn1c(=S)[nH]c(=O)c2[nH]ccc21]

Method a: ®-N-[(1R)-1-[5-chloro-2-[(4-oxo-2-sulfanylidene-2,3,4,5-tetrahydro-1H-pyrrolo[3,2-d]pyrimidin-1-yl)methyl]phenyl]ethyl]-2-methylpropane-2-sulfinamide (140 kg, 82.8% w/w, 264 mol, 1.0 eq), dimethylsulfoxide (556 L, 4.8 vols) and water (140 L, 1.2 vols) were charged to the vessel and the contents were set at 25° ®(R)-(−)-10-Camphorsulfonic acid (123 kg, 528 mol, 2.0 eq) was charged and the resulting solution was heated to 55° C. for 20 hours. The batch was heated to 60° C. and water (70 L, 0.6 vols) was charged over 30 minutes. [(1R)-1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate seed (0.58 kg, 0.5% w/w) was charged and the batch was held for 30 minutes for the seed bed to develop. Water (163 L, 1.4 vols) was charged dropwise at 60° C. and the slurry was held for 1 hour. The slurry was cooled to 50° C. and held for 1 hour, cooled to 40° C. and held for 1 hour, cooled to 20° C. over 4 hours and held at 20° C. for 18 hours. The solid was isolated by filtration and the filter cake was washed with 3:2 DMSO:water (232 L, 2 vols) and ethanol (58 L, 0.5 vols), then dried at 50° C. to yield [(1R)-1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate (142.9 kg, 59% free base assay, 95% yield) as a solid. The seeding in stage 6 method a was to ensure that the solid was more easily isolatable by filtration on a large scale and to improve manufacturability. In an alternative method that was performed, the steps were identical, but a seed was not used and this did not impact overall yield. The seed used in the method above was made using the alternative method a on a scale of 0.2 kg.

¹H NMR (500 MHz, DMSO, 27° C.) 0.72 (3H, s), 1.02 (3H, s), 1.21-1.31 (2H, m), 1.54 (3H, d), 1.77 (1H, d), 1.79-1.87 (1H, m), 1.92 (1H, t), 2.22 (1H, dt), 2.39 (1H, d), 2.59-2.67 (1H, d), 2.88 (1H, d), 4.82 (1H, q), 5.65 (1H, d), 5.79 (1H, d), 6.06 (1H, d), 6.70 (1H, d), 7.29 (1H, dd), 7.34 (1H, dd), 7.70 (1H, d), 8.42 (3H, br s), 11.00-12.97 (2H, br m).

Alternative method b: ®-N-[(1E)-1-[5-chloro-2-[(4-oxo-2-sulfanylidene-2,3,4,5-tetrahydro-1H-pyrrolo[3,2-d]pyrimidin-1-yl)methyl]phenyl]ethylidene]-2-methylpropane-2-sulfinamide (773.4g, 1.77 mol, 1.0 eq.) and 2-methyltetrahydrofuran (3867 mL, 5 vols) were charged to the vessel and cooled to 5° C. Lithium tri-tert-butoxyaluminum hydride (3009 ml, 1 M solution in tetrahydrofuran, 3.01 mol, 1.7 eq.) was charged dropwise and the resulting mixture was held at 10° C. for 3 hours. The mixture was quenched with 28.8% w/w aqueous sodium bisulfate (6190 mL, 8 vols) and the batch was warmed to 20° C. The batch was allowed to separate and the aqueous phase was removed. The organic phase was washed with aqueous sodium chloride solution (2320 mL, 3 vols) followed by 3 M pH 7.2 phosphate buffer solution (1934 mL, 2.5 vols). Dimethyl sulfoxide (3713 mL, 4.8 vol) was charged and the mixture was screened. The contents of the vessel were distilled to approximately 7.5 vols under reduced pressure. Water (365 g, 0.5 vol) was added follow®(R)-(−)-10-Camphorsulfonic acid (838.7g, 3.54 mols, 2.0 eq.) and the resulting solution was heated to 55° C. for ˜16 hours. The batch was heated to 60° C. and water (464.1g, 0.6 vols) was charged over 30 minutes. [(1R)-1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate seed (4.7 g, 0.6 wt %) was charged and the batch was held for 30 minutes for the seed bed to develop. Water (1856 g, 2.4 vols) was charged dropwise at 60° C. over 1.5 hours and the slurry was held for 1 hour. The slurry was cooled to 20° C. over 2.5 hours and held at 20° C. for 15 hours. The solid was isolated by filtration and the filter cake was washed with 1.1:0.9 DMSO:water (1547 mL, 2 vols), water (773 mL, 1 vol) and ethanol (2×773 mL (2×2 vols) then dried at 50° C. to yield [(1R)-1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate (908 g, 59% free base assay, 90% yield) as a solid. The seeding in stage 6 alternative method b was to ensure that the solid was more easily isolatable by filtration on a large scale and to improve manufacturability. In an alternative method that was performed, the steps were identical, but a seed was not used and this did not impact overall yield. The seed used in the method above was made using the alternative method b on a scale of 0.2 kg.

Stage 7/Step (viii): Freebase Formation

1-[[2-[(1R)-1-aminoethyl]-4-chloro-phenyl]methyl]-2-thioxo-5H-pyrrolo[3,2-d]pyrimidin-4-one

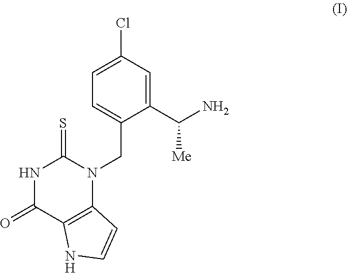 [SMILES: C[C@@H](N)c1cc(Cl)ccc1Cn1c(=S)[nH]c(=O)c2[nH]ccc21]

[(1R)- 1-[5-chloro-2-[(4-oxo-2-thioxo-5H-pyrrolo[3,2-d]pyrimidin-1-yl)methyl]phenyl]ethyl]ammonium (7,7-dimethyl-2-oxo-1-bicyclo[2.2.1]heptanyl)methanesulfonate (137 kg, 238 mol, 1.0 eq) and dimethylsulfoxide (822 L, 6 vols) were charged to the vessel and the temperature adjusted to 22.5° C. The resulting solution was passed through a screening filter. A solution of aqueous ammonium hydroxide (34.3 kg, ˜25% w/w, 511 mol, 2.15 eq.) in water (118 kg, 0.86 vols) was charged dropwise. The batch was warmed to 40° C. and 1-[[2-[(1R)-1-aminoethyl]-4-chloro-phenyl]methyl]-2-thioxo-5H-pyrrolo[3,2-d]pyrimidin-4-one seed (3.97 kg, 5% w/w based on free base) was charged. The resulting slurry was held for 1 hour. Water (129 L, 0.94 vols) was charged over 3.5 hours and the slurry was held for 2 hours. Water (548 L, 4 vols) was charged over 7 hours. The slurry was held for 1 hour before being cooled to 22.5° C. over 1 hour. The batch was held for 8 hours and then isolated by filtration. The filter cake was washed with 1:1 dimethylsulfoxide:water (274 L, 2 vols) followed by washing three times with ethanol (3×274 L, 2 vols). The product was dried at 50° C. to yield 1-[[2-[(1R)-1-aminoethyl]-4-chloro-phenyl]methyl]-2-thioxo-5H-pyrrolo[3,2-d]pyrimidin-4-one (82 kg, 99% w/w, 97% yield) as a solid. The seeding in stage 7 was to ensure that the solid was more easily isolatable by filtration on a large scale and to improve manufacturability. In an alternative method that was performed, the steps were identical, but a seed was not used and this did not impact overall yield. The seed used in the method above was made using the alternative method on a scale of 0.2 kg.

¹H NMR (500 MHz, DMSO, 27° C.) 1.31 (3H, d), 3.37 (2H, br s), 4.36 (1H, q), 5.65 (1H, d), 5.81 (1H, d), 6.03 (1H, d), 6.58 (1H, d), 7.10 (1H, dd), 7.2 (1H, d), 7.64 (1H, d). Exchangeable protons not seen.

All references cited herein, including patents, patent applications, papers, text books, and the like, and the references cited therein, to the extent that they are not already, are hereby incorporated herein by reference in their entirety for all purposes.

1. A process for preparing a compound of Formula (I) comprising at least one of steps (i) to (viii); 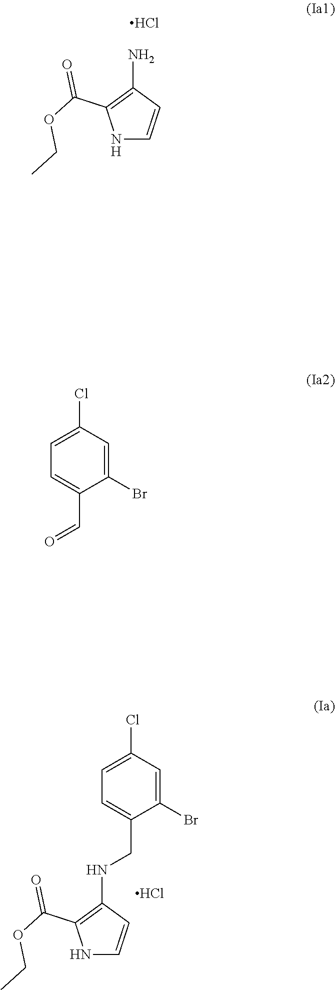 [SMILES: Cl ; CCOC(=O)c1[nH]ccc1N ; O=Cc1ccc(Cl)cc1Br ; CCOC(=O)c1[nH]ccc1NCc1ccc(Cl)cc1Br ; Cl]
(i) forming a compound according to Formula (Ia) from a compound according to Formula (Ia1), or the corresponding freebase or a different salt thereof, and a compound according to Formula (Ia2); 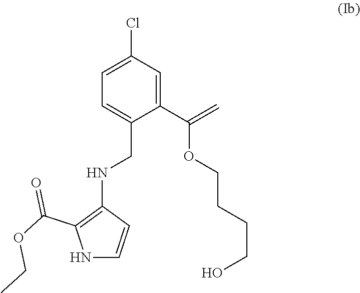 [SMILES: C=C(OCCCCO)c1cc(Cl)ccc1CNc1cc[nH]c1C(=O)OCC]
(ii) forming a compound of Formula (Ib) from a compound of Formula (Ia); 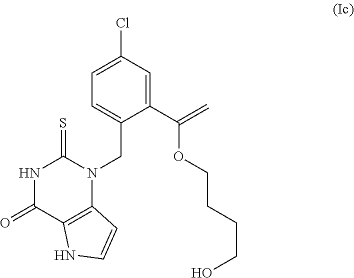 [SMILES: C=C(OCCCCO)c1cc(Cl)ccc1Cn1c(=S)[nH]c(=O)c2[nH]ccc21]
(iii) forming a compound of Formula (Ic) from a compound of Formula (Ib); 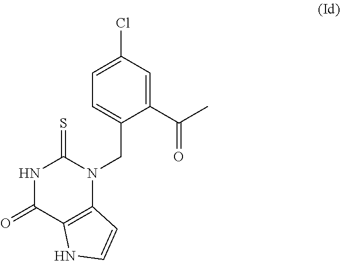 [SMILES: CC(=O)c1cc(Cl)ccc1Cn1c(=S)[nH]c(=O)c2[nH]ccc21]
(iv) forming a compound of Formula (Id) from a compound of Formula (Ic); 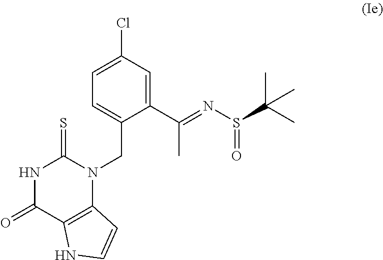 [SMILES: C/C(=N\[S@](=O)C(C)(C)C)c1cc(Cl)ccc1Cn1c(=S)[nH]c(=O)c2[nH]ccc21]
(v) forming a compound of Formula (Ie) from a compound of Formula (Id); 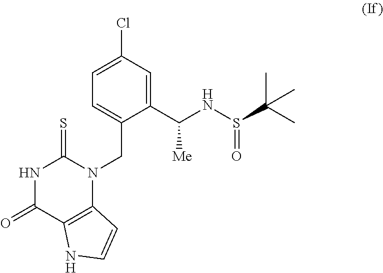 [SMILES: C[C@@H](N[S@](=O)C(C)(C)C)c1cc(Cl)ccc1Cn1c(=S)[nH]c(=O)c2[nH]ccc21]
(vi) forming a compound of Formula (If) from a compound of Formula (Ie); 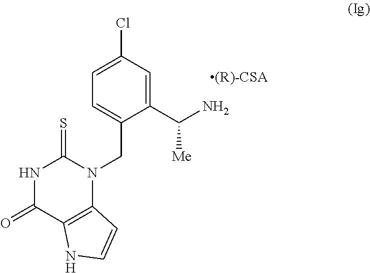 [SMILES: C[C@@H](N)c1cc(Cl)ccc1Cn1c(=S)[nH]c(=O)c2[nH]ccc21]
(vii) forming a compound of Formula (Ig) from a compound of Formula (If); and 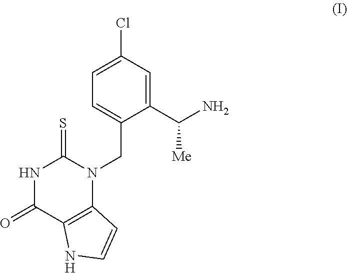 [SMILES: C[C@@H](N)c1cc(Cl)ccc1Cn1c(=S)[nH]c(=O)c2[nH]ccc21]
(viii) forming a compound of Formula (I) from a compound of Formula (Ig).

2. The process for preparing a compound of Formula (I) according to claim 1, comprising at least step (i), wherein step (i) comprises at least the following steps; 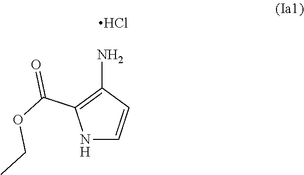
(i-a) breaking the salt of a compound of Formula (Ia1), or the corresponding freebase or a different salt thereof, in the presence of an inorganic base; 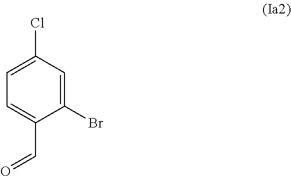
(i-b) reacting the resulting compound of step (i-a) with a compound of Formula (Ia2) under acidic conditions;
(i-c) reducing the resulting imine compound of step (i-b) by reacting with a reducing agent;
and
(i-d) crystallising the resulting compound of Formula (Ia) from step (c) as the hydrochloride salt.

3. The process for preparing a compound of Formula (I) according to claim 1, comprising at least step (ii), wherein step (ii) comprises at least;
(ii-a) reacting a compound of Formula (Ia) with 4-(vinyloxy)butan-1-ol in the presence of a palladium catalyst.

4. The process for preparing a compound of Formula (I) according to claim 1, comprising at least step (iii), wherein step (iii) comprises at least;
(iii-a) reacting a compound of Formula (Ib) with benzoyl isothiocyanate.

5. The process for preparing a compound of Formula (I) according to claim 1, comprising at least step (iv), wherein step (iv) comprises at least;
(iv-a) reacting a compound of Formula (Ic) with a inorganic acid.

6. The process for preparing a compound of Formula (I) according to claim 1, comprising at least step (v), wherein step (v) comprises at least;
(v-a) reacting a compound of Formula (Id) with a chiral sulfinamide reagent in the presence of a dehydrating reagent.

7. The process for preparing a compound of Formula (I) according to claim 1, comprising at least step (vi), wherein step (vi) comprises at least;
(vi-a) reacting a compound of Formula (Ie) with a reducing reagent.

8. The process for preparing a compound of Formula (I) according to claim 1, comprising at least step (vii), wherein step (vii) comprises at least;
(vii-a) reacting a compound of Formula (If) with a chiral resolving agent.

9. The process for preparing a compound of Formula (I) according to claim 1, comprising at least step (viii), wherein step (viii) comprises at least;
(viii-a) reacting a compound of Formula (Ig) with an inorganic base to provide a compound of Formula (I).

10. The process for preparing a compound of Formula (I) according to claim 1 comprising at least step (i).

11. The process for preparing a compound of Formula (I) according to claim 1 comprising at least steps (v) and (vi).

12. The process for preparing a compound of Formula (I) according to claim 1 comprising at least steps (vii) and (viii).

13. The process for preparing a compound of Formula (I) according to claim 1, comprising all of steps (i) to (viii).

14. A compound selected from the group consisting of:

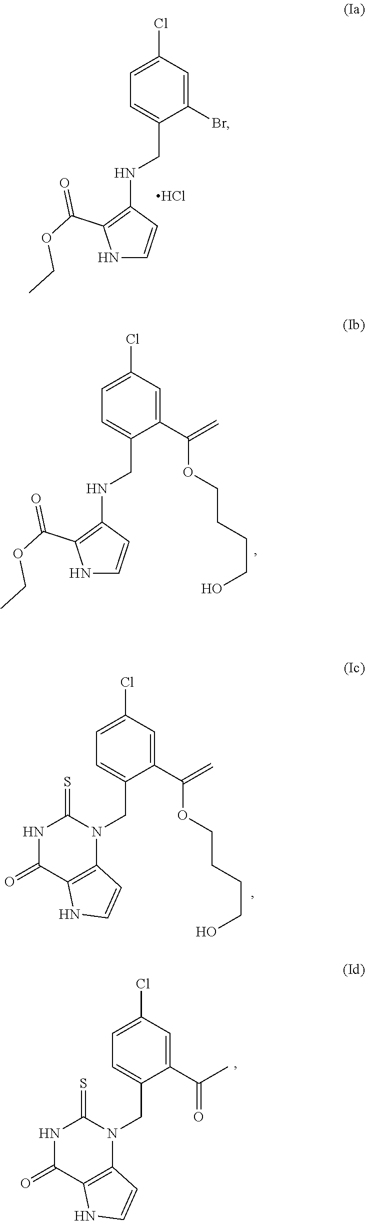 [SMILES: CCOC(=O)c1[nH]ccc1NCc1ccc(Cl)cc1Br ; Cl ; C=C(OCCCCO)c1cc(Cl)ccc1CNc1cc[nH]c1C(=O)OCC ; C=C(OCCCCO)c1cc(Cl)ccc1Cn1c(=S)[nH]c(=O)c2[nH]ccc21 ; CC(=O)c1cc(Cl)ccc1Cn1c(=S)[nH]c(=O)c2[nH]ccc21]

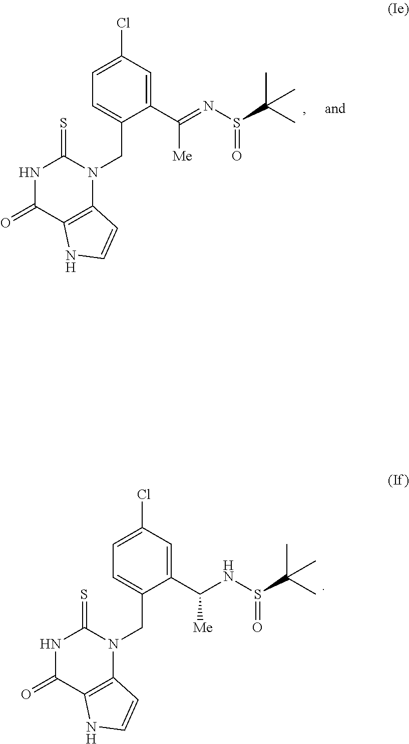 [SMILES: C/C(=N\[S@](=O)C(C)(C)C)c1cc(Cl)ccc1Cn1c(=S)[nH]c(=O)c2[nH]ccc21 ; C[C@@H](N[S@](=O)C(C)(C)C)c1cc(Cl)ccc1Cn1c(=S)[nH]c(=O)c2[nH]ccc21]

15. A compound having the structure: 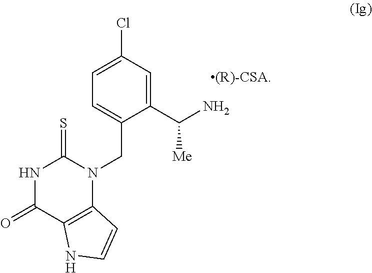 [SMILES: C[C@@H](N)c1cc(Cl)ccc1Cn1c(=S)[nH]c(=O)c2[nH]ccc21]
